(19) United States
(12) Patent Application Publication
(10) Pub. No.: US 2007/0041903 A1
(43) Pub. Date: Feb. 22, 2007

(54) Modified minigastrin analogs for oncology applications

(76) Inventors: Theodosia Maina, Athens (GR); Berthold Artur Noll, Athens (GR); Thomas Behr, Marburg (DE); Martin BEHE, Marburg (DE)

Correspondence Address: GEORGE GEORGELLIS, POST RESTANTE PORTO RAFTI # 1368, MARKOPOULA, MESOGAIA, 19003, GR

(21) Appl. No.: 11/206,757

(22) Filed: Aug. 19, 2005

(51) Int. Cl. A61K 51/00; C07K 7/08

(52) U.S. Cl. 424/1.69; 530/327

(57) ABSTRACT

The invention is directed to modified minigastrin analogs. It further relates to labeling such modified minigastrin analogs with metallic radionuclides. The invention further relates to methods for making such novel modified minigastrin analogs, their labeling with metallic radionuclides and their use in oncology applications as in the targeted diagnostic imaging and staging of CCK-2/gastrin-R-positive neoplasms in man with SPECT (technetium-99m), or PET (technetium-94m), or eventually in targeted radionuclide therapy (rhenium-188).

### TECHNICAL FIELD

The invention relates to methods, compounds, syntheses and preclinical biological evaluation of minigastrin analogs functionalized with 1,4,8,11-tetraazaundecane derivatives. It further relates to labeling such functionalized minigastrin analogs with technetium or rhenium radionuclides preferably with technetium-99m or/and rhenium-188. The invention further relates to the use of such minigastrin analogs in oncology applications.

### BACKGROUND OF THE INVENTION

Metastable technetium-99m (^{99m}Tc) is the “working horse” of nuclear medicine. Over 80% of diagnostic radiopharmaceuticals used today in nuclear medicine clinical studies are ^{99m}Tc-based compounds. D. S. Chem. Rev.; 99 (1999) 2235-2268 Volkert W. A. In: Nicolini M., Bandoli G., Mazzi U., eds. Technetium rhenium and other metals in chemistry and nuclear medicine, 4. Padova: SGEditoriali, (1994) 17-26.

The wide use of ^{99m}Tc is a result of its nearly ideal physical properties. The rather short half-life (t_{1/2}: 6 h) along with the absence of particle emission minimize the radiation dose to the patient. The single energy gamma photons of ^{99m}Tc (140 keV) are most suitable for imaging with currently available nuclear medicine instrumentation (single photon emission computed tomography camera, SPECT camera). Additional advantages of using ^{99m}Tc are its cost-effectiveness and wide availability by means of commercial ⁹⁹Mo-^{99m}Tc generators. The radionuclide is eluted from the ⁹⁹Mo-^{99m}Tc generator in the form of sodium pertechnetate sterile solution (^{99m}TcO₄Na) in physiological saline in a high radionuclidic and radiochemical purity and specific activity sufficient for in vivo receptor targeting applications.

For clinical use ^{99m}Tc must form suitable coordination compounds, that will direct it to the desired target (e.g. neoplastic tissue) while favoring a rapid clearance from non-target tissues of the body.

Other technetium radionuclides, as for example technetium-94m (⁹⁴mTc), have been proposed for developing radiotracers for targeted imaging as well as quantitation of biokinetics employing PET (positron emission tomography) technology. Technetium-94m of high purity is produced today in cyclotrons via different nuclear reactions and target-materials; like enriched ⁹⁴Mo. ⁹⁴mTc has E_{β+} 2.47 MeV, t_{1/2} 52.5 min and a high percentage (72%) of positron emission, which are attractive features for targeted imaging applications using PET cameras Qaim S. M. Nucl. Med. Biol., 27 (2000) 323-328.

On the other hand, rhenium-188 (¹⁸⁸Re) plays an increasingly important role as a therapeutic radionuclide in nuclear medicine. ¹⁸⁸Re is a high energy β emitter with a Eₘₐₓ of 2.1 MeV and a half-life of 17 h (t_{1/2}: 17 h). This metallic radionuclide is available in a high purity and a specific activity sufficient for in vivo receptor targeting by means of commercial ¹⁸⁸W/¹⁸⁸Re generators, from where it is eluted in the form of a sterile sodium perrhenate (¹⁸⁸ReO₄⁻) solution in physiological saline See D. S. Chem. Rev.; 99 (1999) 2235-2268 Volkert W. A. In: Nicolini M., Bandoli G., Mazzi U., eds. Technetium rhenium and other metals in chemistry and nuclear medicine, 4. Padova: SGEditoriali, (1994) 17-26.

In addition to its easy availability and low cost, ¹⁸⁸Re is governed by a chemistry very similar to ^{99m}Tc chemistry, since both of these transition elements belong to the same group of the Periodic Table. Consequently, useful preliminary data concerning the potential use of ¹⁸⁸Re radiopharmaceuticals can be extracted from studies on the respective ^{99m}Tc compounds. A few additional advantages of using ¹⁸⁸Re in the targeted radionuclide therapy of cancer are its capacity to irradiate effectively larger tumor masses via the “cross-fire effect”, as well as its rapid re-oxidation to ¹⁸⁸ReO₄⁻ upon release from the complex. Perrhenate is rapidly excreted into the urine thereby minimizing the radiotoxicity of this therapeutic radionuclide. It should be added, that ¹⁸⁸Re emits gamma photons of 155 keV (15%), which permit direct monitoring not only of in vivo kinetics and localization in the tumor but also response to radiotherapy by means of external imaging devices.

Recent advances in oncology include the application of peptide hormone analogs labeled with diagnostic or therapeutic radionuclides in the targeted diagnostic imaging, staging and radionuclide therapy of cancer. This is partly because several cancer cells overexpress on their surface protein receptors for the respective peptide hormones, as for example somatostatin receptors (sst). Liu S., Edwards D. S. Chem. Rev.; 99 (1999) 2235-2268 and Breeman W. A. P., de Jong M., Kwekkeboom D. J., Valkema R., Bakker W. H., Kooij P. P. M., Visser T. J., Krenning E. P. Eur. J. Nucl. Med., 28 (2001) 1421-1429. Though significant advances have been made an approved radiotherapeutic drug has not yet been released in the market.

The two gastrointestinal peptides gastrin and CCK (cholecystokinin) act both as neurotransmitters in the brain and as regulators of gastrointestinal system function, especially in the stomach, the pancreas and the gallbladder as described in Noble F., Wank S. A., et al., Pharmacol. Rev., 51 (1999) 745-781. Breeman W. A. P., de Jong M., Kwekkeboom D. J., Valkema R., Bakker W. H., Kooij P. P. M., Visser T. J., Krenning E. P. Eur. J. Nucl. Med., 28 (2001) 1421-1429. Jensen R. T., Lemp G. F., Gardner J. D. J. Biol. Chem., 257 (1982) 5554-5559. Reubi J. C., Schaer J. C., Waser B. Cancer Res., 57 (1997) 1377-1386. Reubi J. C., Wasser B., et al. Eur. J. Nucl. Med., 25 (1998) 481-490. Behr T. M., Jenner N., et al. Eur. J. Nucl. Med., 25 (1998) 424-430. Behr T. M., Jenner N., et al. J. Nucl. Med., 40 (1999) 1029-1044. De Jong M., Bakker W. H., et al. J. Nucl. Med., 40 (1999) 2081-2087.

Furthermore, they act as physiological growth factors in the gastrointestinal system or as mitogens in several neoplasms, such as gastrinomas and colon carcinomas. Gastrin and CCK share identical five amino acid sequence in their C-terminal, which constitutes their biologically active site. Their actions are elicited after binding to specific G-protein coupled receptors, which are located on the cell membrane of target-cells. These receptors comprise two subtypes, the CCK-1 and CCK-2 receptors, which can be pharmacologically distinguished by their low (CCK-1-R) or high (CCK-2) affinity to gastrin. Both CCK-1-Rs and CCK-2/gastrin-Rs are found in several physiological tissues. Thus, while CCK-1-Rs are located in the gallbladder, the pancreas and the brain, CCK-2/gastrin-Rs are mainly found in the gastric mucosa and the brain. Of particular interest is the CCK-2/gastrin-R overexpression on the surface of several type neoplastic cells. Recent studies suggest that CCK-2/gastrin-R are expressed in high incidence (>90%) in medullary thyroid carcinoma (MTC), in (˜60%) small cell lung cancer (SCLC), in astrocytomas (65%) and stromal ovarian cancers (100%). Other cancer types, like gastroenteropancreatic tumors (GEP tumors), breast, endometrial and ovarian adenocarcinomas, express these receptors at a lower incidence. Noble F., Wank S. A., et al., Pharmacol. Rev., 51 (1999) 745-781. Jensen R. T., Lemp G. F., Gardner J. D. J. Biol. Chem., 257 (1982) 5554-5559. Reubi J. C., Schaer J. C., Waser B. Cancer Res., 57 (1997) 1377-1386. Reubi J. C., Wasser B., et al. Eur. J. Nucl. Med., 25 (1998) 481-490.

Of particular interest is the high incidence CCK-2/gastrin-R overexpression in MTCs. The high diagnostic sensitivity and accuracy of the pentagastrin test in the detection of neoplastic C cells, at much lower levels than those detected applying conventional morphological imaging methods, provide further evidence for the high incidence and high density expression of CCK-2/gastrin-Rs in human MTCs. It is worth noticing, that in contrast to other neuroendocrine tumors, sst expression and consequently the uptake of [¹¹¹In]OctreoScan by MTC are very low. Furthermore, recent studies report that the density of sst expression declines as the disease progresses. Thus, while the sst is expressed at high densities in well differentiated MTCs, in clinically aggressive and rapidly advancing MTCs these receptors are absent. Reubi J. C., Schaer J. C., Waser B. Cancer Res., 57 (1997) 1377-1386. Reubi J. C., Wasser B., et al. Eur. J. Nucl. Med., 25 (1998) 481-490. Behr T. M., Jenner N., et al. Eur. J. Nucl. Med., 25 (1998) 424-430. Behr. T. M., Jenner N., et al. J. Nucl. Med., 40 (1999) 1029-1044. De Jong M., Bakker W. H., et al. J. Nucl. Med., 40 (1999) 2081-2087. Bihi M., Behr T. M. Biopolymers 66 (2002) 399-418. Reubi J. C., Chayvialle J. A., Franc B., et al. Lab. Invest. 64 (1991) 567-573. Kwekkeboom D. J., Reubi J. C., Lamberts S. W. J., et al. J. Clin. Endocrinol. Metab. 76 (1993) 1413-1417.

As a consequence, there is currently strong interest in the development of radiolabeled gastrin and CCK analogs for application in the in vivo imaging or/and radionuclide therapy of primary and metastatic CCK-2/gastrin-R-positive tumors in man, especially of MTCs.

Given that these applications concern primarily metallic radionuclides (e.g. ^{99m}Tc, ^{94m}Tc, ¹⁸⁸Re), the parent and biologically active molecule has to be properly modified to ensure effective binding of the radiometal without affecting the biological activity of the compound. This is usually achieved by covalent coupling of a suitable chelator (e.g. open chain tetraamine system) at a selected position (usually at the N-terminal) of the peptide motif. Selection of chelator type most suitable for the metallic radionuclide in question as well as of the correct coupling site play a key-role in the success of the present invention.

Advantages of the Invention Versus Currently Applied Methods

As detailed in the previous section, in vivo targeting of CCK-2/gastrin-R-expressing tumors, especially MTCs, in man using radiolabeled gastrin and/or CCK analogs for diagnostic or therapeutic purposes has been proposed by several investigators. In fact, a few such analogs have already been tested in experimental animal models or even in the human.

Most of these analogs, however, are modified with DTPA (DTPA=diethylenetriaminepentaacetic acid) or DOTA (DOTA=1,4,7,10-tetraazacyclododecane-1,4,7,10-tetraacetic acid) chelators and are therefore unsuitable for labeling with technetium. On the other hand, ^{99m}Tc is still the gold standard of diagnostic nuclear medicine due to ideal nuclear characteristics, easy availability and cost effectiveness. Furthermore, the radionuclide ^{94m}Tc is attractive for diagnostic applications employing PET technology. Interestingly enough, there exists a convenient therapeutic radionuclide counterpart for ^{99m}Tc as well, namely ¹⁸⁸Re. The latter is commercially available at a relatively low cost through a commercial generator.

Recently, a CCK-8 analog modified with the tetradentate PhosGC chelator—a PNNS donor atom set—has been reported, which could be labeled with ^{99m}Tc. Aloj L., Panico M., Caraco C., et al. Cancer Biother. Radiopharm., 19 (2004) 93-98. Despite its ability to interact with the CCK-2/gastrin-R (K_{d} 20-40 nM), ^{99m}TcPhosGC-CCK-8 exhibited a very high hepatobiliary excretion in mice and was therefore considered unsuitable for further validation in patients.

In view of the above, the present invention provides a synthesis method for making three novel minigastrin (MG) analogs functionalized with open chain tetraamine derivatives for effective binding of ^{99m}Tc, ^{94m}Tc or ¹⁸⁸Re, affording high specific activity radiopeptides [^{99m}Tc/^{94m}Tc/¹⁸⁸Re]Demogastrin 1-3. These analogs have also been fully characterized and have been shown to be useful for applications in clinical oncology both in vitro and in experimental animal models.

The [(D)Glu¹]MG sequence was built on a resin applying solid phase peptide synthesis (SPPS) techniques. The respective Boc-protected 1,4,8,11-tetraazaundecane derivative was coupled directly or through a spacer to the N-terminal. The N₄-functionalized peptides were deprotected and cleaved from the resin with TFA and were purified by HPLC affording the following conjugates: N₄-(D)Glu-Glu-Glu-Glu-Glu-Glu-Ala-Tyr-Gly-Trp-Met-Asp-Phe-NH₂, Demogastrin 1, and N₄-Gly-(D)Glu-Glu-Glu-Glu-Glu-Glu-Ala-Tyr-Gly-Trp-Met-Asp-Phe-NH₂, Demogastrin 2, and N₄-p-CH₂C₆H₄NHCOCH₂OCH₂CO-(D)Glu-Glu-Glu-Glu-Glu-Glu-Ala-Tyr-Gly-Trp-Met-Asp-Phe-NH₂, Demogastrin 3. The products were tested for purity and characterized by HPLC, UV/Vis and ES-MS spectroscopy.

The radiopeptides show clear advantages over the previously reported CCK-2/gastrin-R-affine radioligands, as detailed below:

First, they form nearly quantitatively by means of the open chain tetraamine which effectively binds technetium producing stable and high specific activity radiopeptides of defined structure, confirmed with chromatographic comparison with Re-authentic samples prepared macroscopically and characterized by MALDI-TOF. The convenience of using ^{99m}Tc-based radiopeptides in a clinical setting is very high due to logistic, cost and imaging quality considerations that were outlined in the previous section.

Second, all analogs exhibited a high binding affinity for the CCK-2/gastrin-R during saturation binding experiments in AR4-2J cell membrane homogenates expressing CCK-2/gastrin-Rs. After interaction with the CCK-2/gastrin-R the radiopeptides migrate rapidly and in a high percentage in the intracellular compartment of AR4-2J cells. During biodistribution in healthy mice [^{99m}Tc]Demogastrin 1-3 were rapidly cleared from the body of mice into the urine via the kidneys and the urinary system showing minimal hepatobiliary excretion. By in vivo blocking experiments it was shown that the localization of radioactivity in the gastric mucosa, where CCK-2/gastrin-Rs are normally located, was specific, while a non-specific uptake and retention was not observed for any other organ or tissue. Furthermore, metabolic studies in mice demonstrated that [^{99m}Tc]Demogastrin 1-3 are stable in mouse plasma. In the kidneys the radiopeptides are degraded and are excreted in the urine in the form of hydrophilic metabolites. After injection in athymic mice bearing AR4-2J experimental tumors [^{99m}Tc]Demogastrin 1-3 demonstrated specific uptake in the tumor. In else, the new compounds ideally combine the characteristics of high and specific target localization with a very favorable for imaging application in vivo profile, reported for the first time for a ^{99m}Tc-based gastrin deriving radioligand. In fact, these qualities were confirmed in the human during validation of new analogs currently in progress in a small number of MTC patients illustrating the suitability of the new molecules for application in nuclear oncology.

### SUMMARY OF THE INVENTION

One aspect of the invention is directed to minigastrin analogs modified with 6-R-1,4,8,11-tetraazaundecane or 1,4,8,11-tetraazaundecane derivatives modified minigastrin having the formula: (H₂NCH₂CH₂NHCH₂)₂CH—R—X—HN—(D)Glu-Glu-Glu-Glu-Glu-Alu-Ala-Tyr-Gly-Trp-Met-Asp-Phe-NH₂, wherein R=CO or p-CH₂C₆H₄NH—COCH₂OCH₂CO and X=0 or Gly.

Another aspect of the invention is directed to labeling such inventive modified minigastrin analogs with suitable metallic radionuclides. Preferably, the metallic radionuclides are technetium or rhenium and more preferably are selected from the group consisting of ^{99m}Tc, ^{94m}Tc and ¹⁸⁸Re.

Yet another aspect of the invention is directed to a synthesis method for making such inventive modified minigastrin analogs. More particularly, the invention relates to a method of synthesis on the solid support of minigastrin analogs of the type: (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO or p-CH₂C₆H₄NH—COCH₂OCH₂CO and X=0 or Gly, comprising: a) building of the amino acid chain X—HN—(D)Glu-Glu-Glu-Glu-Glu-Glu-Ala-Tyr-Gly-Trp-Met-Asp-Phe-NH₂, wherein X=H or Gly, on the solid support following Fmoc/Boc techniques, b) coupling of the respective Boc-protected tetraamine chelator precursor (Boc-HN—CH₂CH₂N(Boc)CH₂)₂CH—R, wherein R=CO, or p-CH₂C₆H₄NH—COCH₂OCH₂—CO, at the N-end amino acid ((D)Glu or Gly) of the immobilized peptide sequence employing a coupling reagent, preferrably HATU (hexafluorophosphate o-(7-azabenzotriazolyl-1,1,3,3-tetramethyluronium) in alkaline medium, c) deprotection and release of N₄-peptide conjugates (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO, or p-CH₂C₆H₄NH—COCH₂OCH₂CO and X=0 or Gly, from the resin using trifluoroacetic acid (TFA) and d) purification and isolation of products with chromatography methods.

Yet another aspect of the invention is directed to labeling such modified inventive minigastrin analogs with metallic radionuclides, preferably technetium or rhenium and more preferably ^{99m}Tc, ^{94m}Tc, and ¹⁸⁸Re. The labeling method comprises binding of the metallic radionuclide to the modified minigastrin analog such binding being performed in aqueous medium at alkaline pH in the presence of a transfer ligand such as citrate and a reducing agent. Preferably the reducing agent is bivalent tin. Other suitable transfer agents and reducing agents may also be used.

The invention further relates to the use of such labeled modified minigastrin analogs in oncology applications. More particularly, one aspect of the invention is directed to a method for using modified minigastrin analogs of the type: (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO or p-CH₂C₆H₄NH—COCH₂OCH₂CO and X=0 or Gly, which have been labeled with metallic radionuclides, for the preparation of a radiodiagnostic product for application in the scintigraphic imaging of CCK-2/gastrin-R-positive neoplasms. Preferably, the modified minigastrin analogs are labeled with technetium or rhenium, and more preferably with a metallic radionuclide selected from the group comprising ^{99m}Tc or ^{94m}Tc and ¹⁸⁸Re.

An additional aspect of the invention is directed to the radiochemical study of the technetium-99m (or other technetium radionuclides) or/and rhenium-188 (or other rhenium radionuclides) compounds which are produced from the above steps, determining labeling yields, radiochemical purity and in vitro stability.

Yet another aspect of the invention is the preclinical evaluation of compounds which are produced from the above:
in cell lines expressing the CCk-2/gastrin receptor (CCK-2/gastrin-R)
in experimental animal models
   and the study of their metabolism.

Yet another aspect of the invention relates to a method of using the inventive minigastrin analogs for the preparation of a radiopharmaceutical for application in the radionuclide therapy of CCK-B/gastrin-R-positive neoplasms. Preferably the metallic radionuclides in said minigastrin analogs are selected from the group consisting of technetium and rhenium and more preferably from the group consisting of ^{99m}Tc, ^{94m}Tc, and ¹⁸⁸Re. Especially advantageous in this application is the use of minigastrin analogs labeled with ¹⁸⁸Re.

### BRIEF DESCRIPTION OF THE DRAWINGS

FIG. 1: Formulae of Demogastrin 1-3

FIG. 2: [^{99m}Tc]Demogastrin 2, typical radiochromatogram of [^{99m}Tc]Demogastrin 2.

FIG. 3: Representative saturation binding curve of [^{99m}Tc/^{99g}Tc]Demogastrin 2 to the CCK-2/gastrin-R in AR4-2J cell membranes.

FIG. 4: Internalization rate of [^{99m}Tc]Demogastrin 1-3 in AR4-2J cells at 37IC.

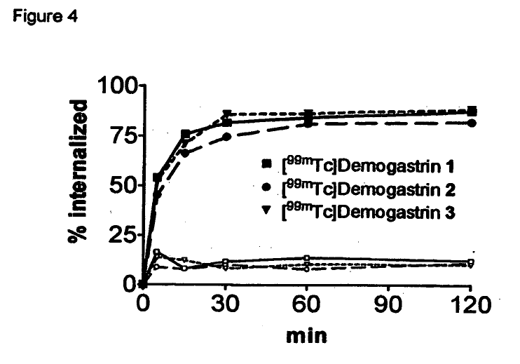

FIG. 5: Biodistribution data of [^{99m}Tc]Demogastrin 1 (A), [^{99m}Tc]Demogastrin 2 (B) and [^{99m}Tc]Demogastrin 3 (C) in healthy mice as % ID/g. Bl=blood, Li=liver, He=heart, Ki=kidneys, St=stomach, In=intestines, Sp=spleen, Mu=muscle, Lu=lungs.

FIG. 6: Degradation of [^{99m}Tc]Demogastrin 1-3 in mouse plasma (A) and kidney homogenates (B). The faster degradation of [^{99m}Tc]Demogastrin 2 in the kidneys favors a faster renal clearance into the urine.

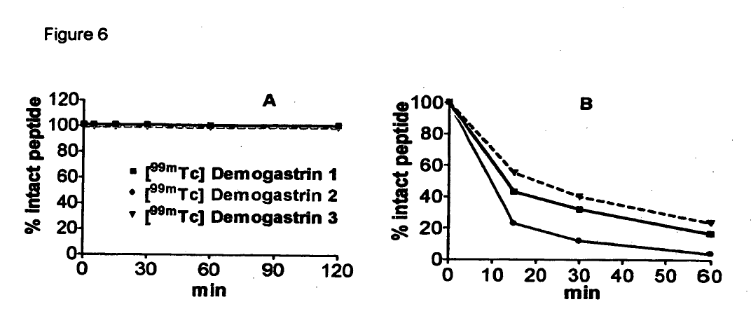

FIG. 7: Biodistribution of [^{99m}Tc]Demogastrin 1-3 in AR4-2J experimental tumor bearing mice as % ID/g. Bl=blood, Li=liver, He=heart, Ki=kidneys, St=stomach, In=intestines, Sp=spleen, Mu=muscle, Lu=lungs, Tu=tumor.

### DETAILED DESCRIPTION OF THE INVENTION

### Experimental

### Synthesis of Peptide Conjugates

Synthesis of Demogastrin 1-3 proceeded after building of the amino acid [(D)Glu¹]MG ((D)Glu-Glu-Glu-Glu-Glu-Glu-Ala-Tyr-Gly-Trp-Met-Asp-Phe-NH₂) sequence on the solid support following Fmoc/Boc methods. Subsequently, (N, N′, N″,N′″-tetra-(tert-butocarboxycarbonyl)-6-R-1,4,8,11-tetraazaundeca-ne, wherein R=COOH, p-CH₂C₆H₄NHCOCH₂OCH₂COOH) was coupled to the N-terminal of the resin-immobilized chain, which for Demogastrin 2 was elongated by a Gly residue (Gly⁰), using a suitable coupling reagent, such as HATU (hexafluorophosphate o-(7-azabenzotriazolyl-1,1,3,3-tetramethyluroni-um) in alkaline medium. The N₄-functionalized peptides were deprotected and released from the resin by reaction with trifluoroacetic acid (TFA). The end products ((H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO, or p-CH₂C₆H₄NH—COCH₂OCH₂CO and X=0 or Gly) were collected after purification using chromatographic methods. Formation and purity of three N₄-modified minigastrin analogs were verified by high performance liquid chromatography (HPLC) and electrospray mass spectroscopy (ES-MS) methods.

### Labeling of Minigastrin Conjugates with ^{99m}Tc

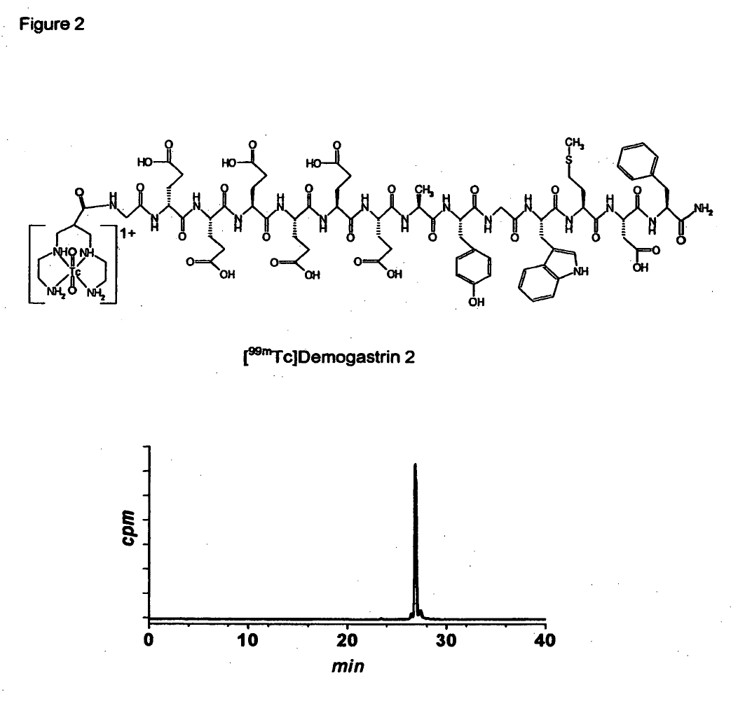

In a vial containing 0.5 M phosphate buffer pH 11.5 (50 μL) the following reagents are added: 0.1 M sodium citrate solution (5 μL), ⁹⁹Mo/^{99m}Tc generator eluate containing Na^{99m}TcO₄ (415 μL, 10-20 mCi), Demogastrin stock solution (15 nmol, 15 μL) and a freshly prepared SnCl₂ solution in ethanol (15 μL, 30 μg). The mixture is left to react for 30 min at ambient temperature and then neutralized (pH 7.0) by adding 1 M HCl. Labeling yields are monitored by radioanalytical HPLC. The chemical formula of [^{99m}Tc]Demogastrin 2 along with a representative radiochromatogram are shown in FIG. 2.

### Saturation Binding Assays of [^{99m}Tc/^{99g}Tc]Demogastrin 1-3 vs. the CCK-2/Gastrin-R

Saturation binding experiments with [^{99m}Tc/^{99g}Tc]Demogastrin 1-3 were performed in AR4-2J cell membrane homogenates. For these assays [^{99m}Tc/^{99g}Tc]Demogastrin 1-3 were prepared following a protocol similar to that used for labeling Demogastrin 1-3 with ^{99m}Tc but using a higher amount of ^{99g}Tc and reductant. [^{99m}Tc/^{99g}Tc]Demogastrin 1-3 were purified via RP-HPLC and two series of triplicates of different radioligand concentrations were prepared. Briefly, to each assay tube containing 50 μL binding buffer (50 mM HEPES pH 7.6 with 0.3% BSA, 10 mM MgCl₂, 14 mg/L Bacitracin), 50 μL radioligand solution of the desired concentration were added, followed by 200 μL AR4-2J cell membrane homogenate corresponding to 100 μg protein. Samples were incubated for 1 h at 37IC and the assay terminated by addition of 10 mL chilled buffer (10 mM HEPES, 150 mM NaCl, pH 7.6) and rapid filtration over glass fiber filters (Whatman GF/B, impregnated in 0.3% BSA) on a Brandel Cell Harvester. Non-specific binding was the binding achieved in the presence of 1 μM [(D)Glu¹]MG.

### Internalization Experiments of [^{99m}Tc]Demogastrin 1-3 in CCK-2/Gastrin-R-Positive Cells

For internalization experiments AR4-2J cells expressing the CCK-2/gastrin-R in sufficient densities were seeded in Ø 35 mm well-plates and incubated at 37IC with [^{99m}Tc]Demogastrin 1-3 (250,000 cpm) for 5, 15, 30, 60 and 120 min time intervals. After reaction with acid buffer pH (50 mM Gly, 0.1 M NaCl, pH 2.8) for 10 min at room temperature samples were collected and their radioactivity content measured (membrane bound activity). After lyzing the cells with 1 N NaOH solubilized cells were collected and measured for radioactivity (internalized activity). Non-specific internalization was considered that in the presence of 1 μM [(D)Glu¹]MG.

### Biodistribution Experiments of [^{99m}Tc]Demogastrin 1-3 in Healthy Mice

For biodistribution experiments male swiss albino mice (30±5 g) were used in groups of four. Each animal was injected through the tail vein with [^{99m}Tc]Demogastrin 1-3 solution in phosphate buffer saline pH 7.4 (100 μL, 2.5-4 μCi). Animals were sacrificed by cardiac puncture while under a slight ether anesthesia at 30 min, 1, 2 and 4 h post injection (pi). Samples of urine, blood as well as organs of interest were immediately collected, weighed and measured for radioactivity in an automated gamma counter. In a separate group of animals 250 μg [(D)Glu¹]MG were intravenously injected along with test radiopeptide (blocked animals). These animals were sacrificed 1 h pi. Biodistribution data were calculated as percent injected dose per organ (% ID/organ) or per gram (% ID/g) employing a known algorithm with the aid of suitable standards.

### Metabolic Experiments

Urine collected 30 min after injection of test radiopeptide (0.5-1 mCi, 200 μL) was analyzed by HPLC.

Fresh plasma was collected after centrifugation of mouse heparinized blood for 15 min at 5000 rpm and incubated with [^{99m}Tc]Demogastrin 1-3 at 37IC. Samples collected at 5, 15, 30 min and 1 h were analyzed by HPLC. Kidneys were excised and homogenized immediately by an Ultra-Turrax T25 homogenizer for 30 sec in 50 mM Tris, 0.2 M sucrose pH 7.4 buffer at 4IC. Homogenates were incubated at 37IC with test radiopeptides for 15, 30 min and 1 h and aliquots were treated with ethanol, centrifuged and analyzed by HPLC.

### Biodistribution Experiments of [^{99m}Tc]Demogastrin 1-3 in Experimental Tumor Bearing Athymic Mice

For biodistribution experiments female Swiss nu/nu mice 6 weeks of age were used. Animals were inoculated in their flanks with a 0.6-0.8×10⁷ AR4-2J cell suspension and 2-3 weeks later developed well visible tumors and biodistribution was conducted. For this study groups of four animals each were used, which were sacrificed at 1 and 4 h after injection of test radiopeptide. A parallel group of animals sacrificed at 4 h pi received 250 μg [(D)Glu¹]MG along with the radiopeptide (blocked animals). Biodistribution was performed as described above.

### Results—Discussion

### Synthesis of Demogastrin 1-3

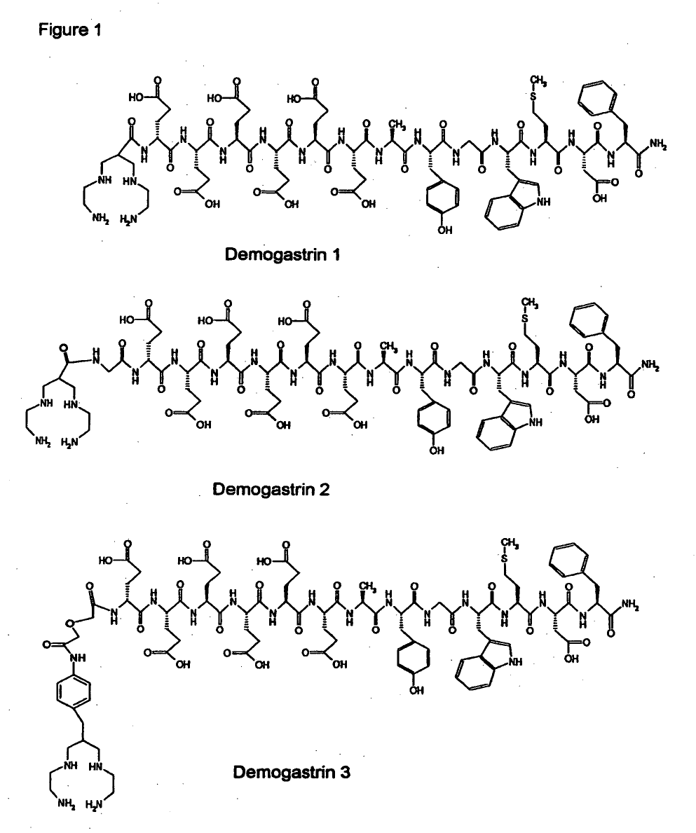

The [(D)Glu¹]MG sequence (R′-HN-(D)Glu-Glu-Glu-Glu-Glu-Glu-Ala-Tyr-Gly-Trp-Met-Asp-Phe-NH₂, R′=H or Gly⁰) was built on the solid support [14] and the respective Boc-protected chelator, ((Boc-N )₄-6-R-1,4,8,11-tetraazaundecane, R=COOH or p-CH₂C₆H₄NHCOCH₂OCH₂—COOH) was coupled at the N-terminal. The N₄-functionalized peptide analogs (Demogastrin 1-3, FIG. 1) after purification were identified by HPLC, UV/Vis and ES-MS spectroscopy.

### Labeling of Demogastrin 1-3 with ^{99m}Tc

Labeling with ^{99m}Tc was performed in phosphate buffer pH 11.5 using citrate as transfer ligand and SnCl₂ for pertechnetate reduction. As shown by HPLC analysis, a >95% labeling yield was typically reached in high specific activities (>10 mCi ^{99m}Tc with 15 nmol peptide).

### Binding Affinity of [^{99m/99g}Tc]Demogastrin 1-3 for the CCK-2/Gastrin-R

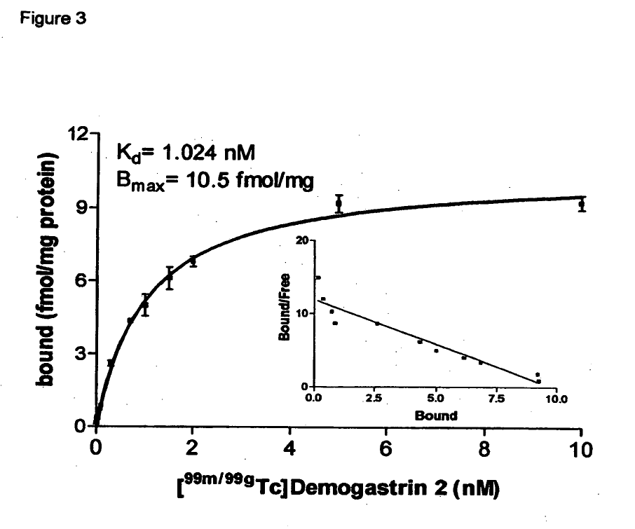

The binding affinity of radiopeptides for the CCK-2/gastrin-R was determined during saturation binding experiments in AR4-2J cell membrane homogenates. All analogs exhibited a high affinity binding to the CCK-2/gastrin-R with K_{d} values in the low nM range, as shown in FIG. 3 for [^{99m}Tc/^{99g}Tc]Demogastrin 2.

### Internalization of [^{99m}Tc]Demogastrin 1-3 in CCK-2/Gastrin-R-Positive Cells

After binding to the CCK-2/gastrin-R on the membrane of AR4-2J cells, [^{99m}Tc]Demogastrin 1-3 migrate rapidly and in a high percentage in the intracellular compartment of AR4-2J cells. The ˜85% internalization maximum is reached within 30 min, while in the first 15 min already a ˜80% internalization is typically observed. The internalization maximum remains high up to 2 h of the study. In all cases non-specific internalization was negligible.

### Biodistribution of [^{99m}Tc]Demogastrin 1-3 in Healthy Mice

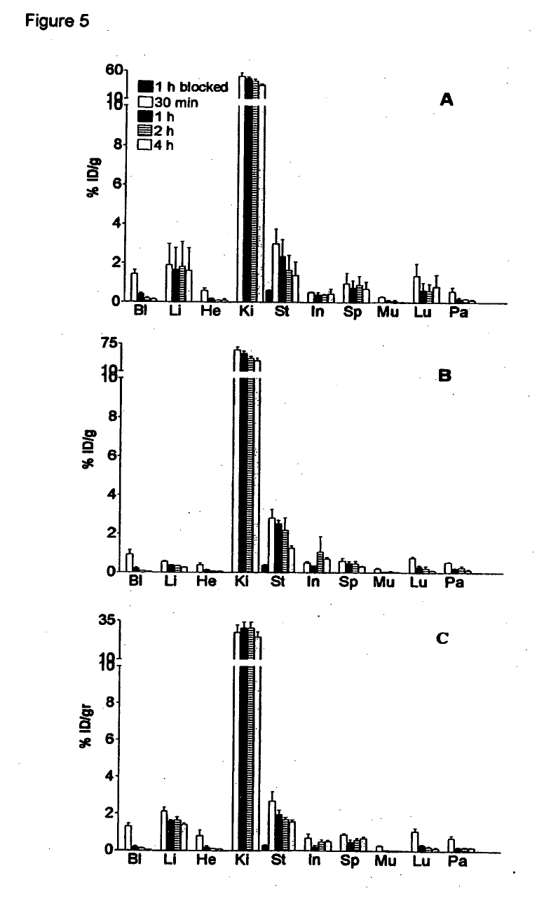

After intravenous injection in healthy mice, [^{99m}Tc]Demogastrin 1-3 are rapidly cleared from the body of animals via the kidneys and the urinary system into the urine. At the same time, the analogs are localized at the gastric mucosa, where the in vivo binding sites of native gastrin are located. This gastric uptake is significantly reduced in the animals that received a high dose [(D)Glu¹]MG along with the radiopeptide (blocking experiments). Biodistribution data as % ID/g are summarized in FIG. 5.

### Metabolism

Analysis of urine collected 30 min after intravenous administration of radiopeptides revealed that they are all excreted in the form of hydrophilic metabolites.

All analogs remain stable during incubation in mouse plasma. Incubation of radiopeptides in mouse kidney homogenates at 37IC has shown that they are converted to hydrophilic products. Resistance to degradation follows the rank: [^{99m}Tc]Demogastrin 3 >[^{99m}Tc]Demogastrin 1>>[^{99m}Tc]Demogastrin 2. The faster degradation of [^{99m}Tc]Demogastrin 2 in the kidneys favors a faster renal clearance into the urine.

### Biodistribution of [^{99m}Tc]Demogastrin 1-3 in Experimental Tumor Bearing Athymic Mice

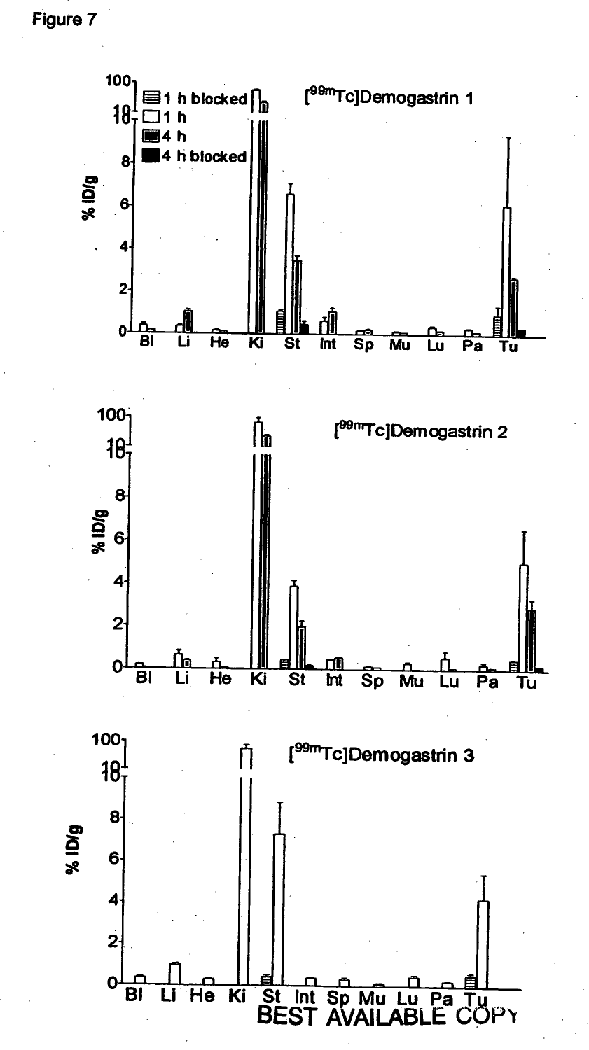

Biodistribution data of [^{99m}Tc]Demogastrin 1-3 in mice bearing a CCK-2/gastrin-R-positive experimental tumor are presented as % ID/g in FIG. 7

Parallel to their rapid clearance from the body of experimental animals [^{99m}Tc]Demogastrin 1-3 are localized rapidly and in a high percentage in the stomach and the CCK-2/gastrin-R-positive tumor, where they remain in satisfactory levels up to the 4 h pi of the study. This uptake is specific, given that it is >90% reduced in the groups of blocked animals. Particularly favorable tumor to background ratios are achieved by [^{99m}Tc]Demogastrin 2. This analog is undergoing clinical evaluation in MTC patients.

New minigastrin peptide analogs, carrying an open chain tetraamine chelator on their N-terminal for stable binding of ^{99m}Tc (diagnosis) or/and ¹⁸⁸Re (therapy), were synthesized on the solid support and characterized (Demogastrin 1-3). Demogastrin 1-3 can be easily labeled with ^{99m}Tc under mild conditions in high yields and high specific activities, sufficient for in vivo receptor targeting applications (CCK-2/gastrin-R).

The radiolabeled analogs ([^{99m}Tc]Demogastrin 1-3) are hydrophilic and are excreted very rapidly from the body of mice predominantly over the kidneys and the urinary system into the urine.

The radiopeptides are localized in the gastric mucosa as a result of specific interaction with the CCK-2/gastrin-Rs, as demonstrated by in vivo blocking experiments. [^{99m}Tc]Demogastrin 1-3 are stable in murine plasma while degrading in the kidneys to hydrophilic products which are excreted in the urine.

[^{99m}Tc]Demogastrin 1-3 are localized rapidly and specifically in experimental CCK-2/gastrin-R-positive tumors, where they are retained for satisfactory time periods.

The above favorable qualities validate the new minigastrin analogs, [^{99m}Tc]Demogastrin 1-3, for application in the scintigraphic detection of CCK-2/gastrin-R-positive neoplasms in man. At the same time they provide a solid basis for the development of the respective ¹⁸⁸Re compounds for targeted radionuclide therapy.

Specifically, the rhenium radionuclide labeled minigastrin analogs of the type: (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO or p-CH₂C₆H₄NH—COCH₂OCH₂CO and X=0 or Gly, can also be used for the preparation of a radiopharmaceutical for application in the radionuclide therapy of CCK-B/gastrin-R-positive neoplasms.

Particularly preferred is the use of the ¹⁸⁸Re labeled minigastrin analogs of the type: (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO or p-CH₂C₆H₄NH—COCH₂OCH₂CO and X=0 or Gly, for the preparation of a radiopharmaceutical for application in the radionuclide therapy of CCK-B/gastrin-R-positive neoplasms.

1. The 6-R-1,4,8,11-tetraazaundecane modified minigastrin analogs of SEQ ID NO:1 or SEQ ID NO:2 of the type: (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO or p-CH₂C₆H₄NH—COCH₂OCH₂CO and X=0 or Gly.

2. The modified minigastrin analogs of claim 1 further comprising metallic radionuclides as labeling agents.

3. The modified minigastrin analogs of claim 1 further comprising a metallic radionuclide selected from the group consisting of ^{99m}Tc, ^{94m}Tc and ¹⁸⁸Re.

4. The method of synthesis on the solid support of minigastrin analogs of SEQ ID NO:1 or SEQ ID NO:2 of the type: (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO or p-CH₂C₆H₄NH—COCH₂OCH₂CO and X=0 or Gly, (SEQ ID NOs:1,2) comprising: a) building of the amino acid chain of SEQ ID NO:1 OR SEQ ID NO:2 of the type X-HN-(D)Glu-Glu-Glu-Glu-Glu-Glu-Ala-Tyr-Gly-Trp-Met-Asp-Phe-NH₂, wherein X=H or Gly, on the solid support following Fmoc/Boc techniques, b) coupling of the respective Boc-protected tetraamine chelator precursor (Boc-HN—CH₂CH₂N(Boc)CH₂)₂CH—R, wherein R=CO, or p-CH₂C₆H₄NH—COCH₂OCH₂—CO, at the N-end amino acid ((D)Glu or Gly) of the immobilized peptide sequence employing a coupling reagent, preferably HATU in alkaline medium, c) deprotection and release of N₄-peptide conjugates (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO, or p-CH₂C₆H₄NH—COCH₂OCH₂CO and X=0 or Gly, from the resin using trifluoroacetic acid (TFA) and d) purification and isolation of products with chromatography methods.

5. The method for labeling the N₄-functionalized molecules of SEQ NO:1 or SEQ ID NO:2 of the type (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO, or p-CH₂C₆H₄NH—COCH₂OCH₂CO and X=0 or Gly, synthesized according to claim 4, with metallic radionuclides

6. The method of claim 4 wherein said metallic radionuclides are technetium or rhenium.

7. The method of claim 4 wherein said metallic radionuclides are selected from the group consisting of ^{99m}Tc, ^{94m}Tc, and ¹⁸⁸Re.

8. The method of claim 4, whereby binding of the metallic radionuclide is conducted in aqueous medium at alkaline pH in the presence of citrate or other transfer ligand and a reducing agent.

9. The method of claim 4, wherein said reducing agent is bivalent tin.

10. A method for using modified minigastrin analogs of SEQ ID NO:1 or SEQ ID NO:2 of the type: (H₂NCH₂CH₂NHCH₂)₂CH—R—X—HN—(D)Glu-Glu-Glu-Glu-Glu-Glu-Ala-Tyr-Gly-Trp-Met-Asp-Phe-NH₂, wherein R=CO or p-CH₂C₆H₄NH—COCH₂OCH₂CO and X=0 or Gly, (SEQ ID NOs 1,2 ) which have been labeled with metallic radionuclides, for the preparation of a radiodiagnostic product for application in the scintigraphic imaging of CCK-2/gastrin-R-positive neoplasms.

11. The method of claim 6 wherein said metallic radionuclides are technetium or rhenium.

12. The method of claim 6 wherein said metallic radionuclides are selected from the group comprising ^{99m}Tc or ^{94m}Tc and ¹⁸⁸Re.

13. (canceled)

14. (canceled)

15. A method of using the minigastrin analogs of claim 2 for the preparation of a radiopharmaceutical for application in the radionuclide therapy of CCK-2/gastrin-R-positive neoplasms.

16. The method of claim 15 wherein said metallic radionuclides in said minigastrin analogs are selected from the group consisting of technetium and rhenium.

17. The method of claim 15 wherein said metallic radionuclides in said minigastrin analogs are selected from the group consisting of ^{99m}Tc or ⁹⁴mTc and ¹⁸⁸Re.

18. The method of claim 15 wherein said minigastrin analogs are labeled with ¹⁸⁸Re


SEQUENCE LISTING (2 sequences)

SEQ ID NO: 1 - protein, 11 aa, artificial sequence
  synthetic peptide
  Xaa (1)..(1): xaa is a D(GLU)
XEEEEEAYWMD

SEQ ID NO: 2 - protein, 12 aa, artificial sequence
  synthetic peptide
  Xaa (2)..(2): Xaa is D(GLU)
GXEEEEEAYWMD